(19) United States
(12) Patent Application Publication
(10) Pub. No.: US 2011/0123592 A1
(43) Pub. Date: May 26, 2011

(54) Biomaterials

(75) Inventors: Molly Stevens, London (GB); Jose Sardinha, Coimbra (PT); Erh-Hsuin Lim, London (GB)

(73) Assignee: IMPERIAL INNOVATIONS LIMITED, London (GB)

(21) Appl. No.: 12/994,606

(22) PCT Filed: May 27, 2009
(86) PCT No.: PCT/GB2009/001327

§ 371 Date: Feb. 9, 2011

(30) Foreign Application Priority Data
May 27, 2008 (GB) 0809592.9

(51) Int. Cl. A61K 47/48; C08G 63/91; A61K 9/00; A61P 43/00

(52) U.S. Cl. 424/423; 525/54.1; 424/78.17

(57) ABSTRACT

The invention relates to a biomaterial having a latent form of a growth factor immobilised thereon, and to a method of producing the biomaterial, comprising immobilising the latent form of the growth factor on the biomaterial. The biomaterial may be used in medicine, such as in tissue regeneration or repair.

The present invention generally relates to the field of biomaterials, which can be used, for example, in tissue engineering. In particular, the invention relates to products and methods for use in tissue engineering or repair, for example, in bone and cartilage tissue engineering or repair. The invention generally relates to the immobilization of a latent form of a growth factor on the surface of a biomaterial, to a process of preparing such a biomaterial, and to methods of using such a biomaterial.

Cartilage degeneration caused by congenital abnormalities or disease and trauma is of great clinical consequence, given the limited intrinsic healing potential of the tissue. Cartilage is an avascular tissue (lacks a blood supply) and its wound-healing response means that especially in adults, there is little or no capacity for the self-repair of eroded articular cartilage. Thus, damage to cartilage (e.g. chondral lesions) is likely to result in an incomplete attempt at repair by local chondrocytes. Despite the relative success of total joint replacement, treatments for repair of cartilage damage are often less than satisfactory, and rarely restore full function or return the tissue to its native normal state. The systemic administration of growth factors to aid cartilage tissue repair is not an option because of the toxicity and pleiotropic effects of these molecules and unphysiologically high doses required for efficacy (Prud'homme & Piccirillo, 2000. J. Autoimmun., 14, 23-42). For articular cartilage damage some techniques to aid repair include resurfacing the defect with periosteum (or perichondrium) transplantation or use of a periosteal flap or biomaterials flap in combination with autologous chondrocyte implantation. Both approaches are mainly limited to very small localized lesions and the nature of the regenerated cartilage (fibrous versus hyaline) is still undetermined. Mosaicplasty is another common approach where osteo-chondral plugs are taken from a non-load bearing surface (normally in the femur) and transplanted into the defect but the technique is associated with morbidity and fibrocartilage can form between the transplanted plugs. In the case of large cartilage defects resulting from trauma or cartilage damage resulting from osteoarthritis (which can be large and unconfined), current treatments are unsuitable and total or partial joint replacement is often performed.

During the last ten years, new tissue engineering concepts have held some promise for the generation of functional tissue substitutes, including cartilage (Ng et al., 2007. Ann Biomed Eng. 35, 11, 1914-23), bone (Fedorovich et al., Tissue Eng Part A. 2008,14, 1, 127-33; Yefang et al., 2007. Int J Oral Maxillofac Surg. 36, 2, 37-45) and many other tissues such as skin (Priya et al., 2008. Tissue Eng Part B Rev., 14, 1, 105-18), neurons, liver, among others (for review see Lee et al., 2008. Tissue Eng Part B Rev., 14, 1, 61-86), by engineering 3 dimensional tissue constructs in vitro for subsequent implantation in vivo or direct in vivo injection/implantation. The basic principle is to use a biocompatible, structurally and mechanically appropriate scaffold seeded with an appropriate cell source, and loading the scaffold with bioactive molecules. to promote cellular differentiation and/or maturation. There have been a number of approaches to engineering tissues, using natural and synthetic biomaterial scaffolds together with allogeneic and autologous sources of mature, progenitor or stem cells (Kim & Recum, 2008, Tissue Eng Part B Rev., 14, 1, 133-47), plus specific inductive growth factors (osteoinductive, chondroinductive, among others) and combinations of these, to drive for example osteogenesis and chondrogenesis.

The 3-dimensional scaffold provides structural support for higher level of tissue organization and remodelling, providing a temporary structure while seeded cells synthesize new natural tissue. Cytokines and growth factors on the other hand play a crucial role in the control of many aspects of cell behaviour including proliferation, migration, matrix production and differentiation of different cell types (Polizzotti et al., 2008. Biomacromolecules. 9, 4, 1084-7; Franzesi et al., 2006. J. Am. Chem. Soc., 128, 47, 15064-5). The culture of cells in scaffolds in culture medium containing different growth factors has induced the differentiation of cells into cartilage and bone, among other tissues. However, these exogenous growth factors are typically added directly to the culture medium (ie. not supplied directly from the scaffold), and thus the differentiation of cells in vitro within this system is not directly translatable to the in vivo situation as an in vitro differentiation step is required. To overcome this, attempts have been made to incorporate free forms of bioactive molecules such as growth factors inside the scaffolds, mainly within injectable hydrogels, for delivery of such agents in vivo in order to achieve some in situ spatial and temporal control of the proliferation/differentiation process (Levenberg et al., 2003, PNAS, 100, 22, 12741-12746). Because the free forms of the growth factors have short half-lives they become ineffective after a short duration (2-3 min for TGFβ1) (Coffey et al., 1987. J. Clin Inv., 80, 750-757; Wakefield et al., 1990. J Clin Inv., 86, 1976-84).

Accordingly, in order to control the release of growth factors in specific locations over prolonged time periods and mitigate their poor pharmacokinetic profiles, attempts have been made to encapsulate one or more bioactive molecules in carriers (mainly based on copolymers of PLA and PGA), which are later incorporated into the scaffold formulation/preparation (Chen et al., 2007. J. Control Release., 12, 118, 1, 65-77). Examples can be found in the literature using single, dual or even multiple release of growth factors, for example for cartilage (Park et al., 2005. Biomaterials, 26, 7095-7103), bone (Lu et al., 2001. J Bone Joint Surg Am., 83, S82-91) to promote angiogenesis (Steffens et al., 2004. Tissue Eng., 10, 1502-1509) among others. However, the approach requires a difficult and burdensome design and optimization process to achieve a time-dependent release of the incorporated biologically active molecules as the carrier molecules (frequently formulated as microparticles) degrade in vivo. The encapsulation process itself, e.g. if involving organic solvents, may also decrease the bioactivity of the growth factors. It should also be noted that the combined effect of multiple growth factors is not always favourable, some negatively affect cartilage yield for example (Veilleux et al., 2005. Osteoarthritis Cartilage, 13, 278-286). Recreating the in vivo regulatory effects of all these signalling molecules is difficult as this depends not only on the chemical properties of the growth factor itself but also on its presentation, dosage and timing of administration.

The present invention relates to a simpler and more biomimetic strategy where the release of one or more growth factors from the scaffold is mediated by cell interaction and cell specific demands in accordance with required phases of cell cycle, nroliferation or differentiation and according to cell type and source. This way the cell itself can “activate” an immobilised latent form of the growth factor as required for multiple cell behaviours and the growth factor remains protected until its requisite activation.

Cytokines and other cellular growth factors are known to regulate the growth and function of cells and tissues in general. They are cell messengers and act in low concentrations (nanomolar to femtomolar) by binding to cell receptors, causing a hormone-like action. These molecules are key modulators of cell proliferation, differentiation and matrix production, among other events (Alsberg et al., 2006. Expert Opin Biol Ther. 6, 9, 847-66). Most cytokines and growth factors are expressed under tight control mechanisms. Their gene expression is regulated by environmentalstimuli such as infection, cell-cell interactions, extracellular matrix composition and interactions with adhesion molecules or via stimulation with other cytokines. However, in some cases, cytokine activity regulation involves the secretion of molecules in a latent form that become “activated” by releasing the cytokine moiety when processes of inflammation, wound healing and tissue repair takes place (Khalil N, 1999. Microbes and Infection, 1, 1255-1263). Many cells produce growth factors in latent form and store them in their extracellular matrix (ECM). Activation can occur at a later time and act on the original cell as an autocrine factor or neighboring cells as a paracrine factor. In this respect, the transforming growth factor beta (TGFβ) family has received most attention because of the broad range of biological processes they can modulate.

We have demonstrated proof of concept of the invention using the latent form of the growth factor Transforming Growth Factor-β1 (TGFβ1). The concept can however be applied to other latent forms of growth factors such as members of the TGFβ superfamily, or growth factors provided in the form of latent fusion proteins.

In a first aspect, the invention provides a biomaterial having a latent form of a growth factor immobilised thereon. The biomaterial may be any material capable of being implanted into a host organism. The biomaterial can be naturally or synthetically nroduced. Biomaterials (also referred to herein as scaffolds) serve a central role in regenerative medicine applications and several basic requirements have been identified (degradation time, pore size and porosity). Scaffolds have been fabricated from a range of natural and synthetic materials, with biodegradable materials being desirable to avoid a second surgical procedure.

Any known biomaterial capable of having a latent form of a growth factor immobilised thereon can be used in the present invention. For example, different materials can be used to immobilize the latent growth factor, such as synthetic polymers and copolymers (e.g. poly-L-lactic acid (PLLA), poly(lactic-co-glycolic acid (PLGA), polyethylene glycol (PEG), polyethylene-co-vinylacetate, and others), natural polymers (e.g. polysaccharides, proteins, proteoglycans, all types of collagen, hyaluronic acid, starch, chitosan, chitin, dextran, pullulan, and others known in the art) or combinations thereof, either degradable or non-degradable, but not limited to these. For example, the biomaterial can comprise a polyester, such as PLLA, PLGA, poly caprol lactone, poly hydroxyl alkanoates, and other polyesters. The biomaterial can be in the form of a gel, sol-gel, hydrogel, membrane, fibrous structures, nano or microfibers, micro or nanowires, porous sponges, woven or non-woven meshes, other known forms, or any combination thereof. The biomaterial can be prepared using different procedures such as gas foaming/particulate, freeze-drying, electrospinning, thermal induced phase separation, injectable scaffolds, but not limited to these. The immobilization can be performed as well onto any type of other material for implantation into the body such as ceramic materials such as hydroxyapatite, soluble glasses and ceramic forms, metallic materials or composite materials, and combinations thereof, including combinations with previous described possibilities.

In the example provided below, electrospun poly-L-lactic acid (PLLA) fibres have been chosen as a scaffold to demonstrate proof of concept because of their biodegradability and US Food and Drug Administration (FDA) approval for clinical use in some devices. Additionally, in vivo and in vitro studies using poly(lactic acid)-based scaffolds have demonstrated the maintenance of chondrocyte phenotype (Mouw et al., 2005. Osteoarthritis Cartilage, 13, 828-836) mainly because of morphological similarities of nanofibers with natural ECM. Thus, the biomaterial is preferably an electrosupun poly-L-lactic acid fibre. However, it should be noted that any of the biomaterials described above can be used in the present invention.

Any growth factor provided in a latent form can be used in the present invention. The growth factor can be any molecule capable of stimulating cell growth, migration, dedifferentiation, redifferentiation or differentiation. For example, the growth factor may be, but is not limited to, TGFβ, epidermal growth factor (EGF), platelet derived growth factor (PDGF), nerve growth factor (NGF), colony stimulating factor (CSF), hepatocyte growth factor, insulin-like growth factor, placenta growth factor); differentiation factor; a cytokine eg. interleukin, (eg. IL1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20 or IL-21, each either α or β), interferon (eg. IFN-α, IFN-β and IFN-γ), tumour necrosis factor. (TNF), IFN-γ inducing factor (IGIF), bone morphogenetic protein (BMP); a chemokine (eg. MIPs (Macrophage Inflammatory Proteins) e.g. MIP1α and MIP1β; MCPs (Monocyte Chemotactic Proteins) e.g. MCP1, 2 or 3; RANTES (regulated upon activation normal T-cell expressed and secreted)) and trophic factors. For example, the growth factor may be selected from the group of TGF-β1 TGF-β2, TGF-β3, TGF-β4, TGF-β5 or any other member of the TGF-β superfamily including activins, inhibins and bone morphogenetic proteins including BMP1, BMP2, BMP3, BMP4, BMP5, BMP6, BMP7. Preferably, the bioactive molecule is derived from the species to be treated e.g. human origin for the treatment of humans.

The present invention is illustrated herein with specific reference to the use of TGFβ, though any growth factor provided in a latent form can be used. Because many cell types express and respond to TGFβ factors there is a complex mechanism for ensuring that TGF-β levels in the ECM are tightly controlled, which involves (besides gene transcription regulation) the storage of the growth factor as a latent molecule. In most cell types, TGFβs are secreted in a latent form consisting of TGFβ and its latency associated peptide (LAP) propeptide dimers, covalently linked to latent TGFβ-binding proteins (LTBPs). TGF-βs are secreted from cells in a latent dimeric complex containing the C-terminal mature TGF-β and its N-terminal pro-domain, LAP (TGF-β latency associated protein) (Roberts and Sporn, 1996. Springer-Verlag, 419-472; Roth-Eicchorn et al., 1998. Hepatology, 28 1588-1596). This precursor structure appears to be shared by all known members of the TGFβ superfamily with the exception of TGFβ4. The two polypeptide chains of proTGF-β associate to form a disulphide bonded dimer. TGF-β is cleaved from its propeptide by furin-like endoproteinase during secretion at RRXR sequence. The LAP propeptide dimer remains associated with the TGF-β dimer by non-covalent interactions, whereas both the mature TGF-β dimer and the LAP dimer are disulphide bonded. The latent TGF-β complex consisting of LAP and TGF-β is referred to as Small Latent TGF-β Complex (SLC). The association between active TGF-β and its LAP propeptide is reversible and involves extensive structural changes in the LAP. The LAP, in addition to protecting TGFβ, contains important residues (RGD) necessary for the interaction with other molecules, namely cell membrane integrins. In most cells LAP is covalently linked to an additional protein, latent TGF-β binding protein (LTBP) which contains several proteinase sensitive sites, providing a way to solubilise the large latent complex (LLC) from the ECM structures. The LTBP is important for the secretion of the complex, folding of TGFβ, targeting the binding to the ECM (mainly elastin fibrils and fibronectin-rich pericellular fibers) and preventing interactions of the TGFβ1 with local matrix proteins. LAP, but not LTBP, is responsible and sufficient for the latency of TGF-β. (Saharinen et al., 1999. Cytokine Growth Factor Rev. 11, 2691-2704).

The activation of the latent TGFβ involves the disruption/dissociation of the non-covalent interaction between the LAP and TGFβ, in order to allow the interaction of the mature peptide with its signalling receptor. Several mechanisms have been proposed to describe (Saharinen, 1999. Cytokine Growth Factor Rev, 10, 99-117) the release of TGFβ, but the process is complex and tissue dependent and so still not entirely clear. All mechanisms involve dissociation of TGF-β1 from LAP-β1 in the soluble SLC and/or the ECM-bound LLC. Although LTGFβ can be activated by transient acid, base, heat, or chaotrophic agents like urea in a test tube, in vivo,—oteolytic cleavage appears to be the most prominent process of TGF-β activation involving different proteases (Bone Morphogenetic Protein (BMPs), matrix metalloproteases (MMPs), plasmin, thrombospondin 1, leukocyte elastase, mast cell chymase, among others) that cleave the LTBP at a protease-sensitive hinge region and target the cleaved complex to the cell surface (Taipale et al., 1992. J Biol Chem., 267,25378-25384). The truncated LLC and the SLC can then be subjected to different mechanisms of in vivo activation: a) degradation of the LAP by proteases; b) induction of a conformational change in the LAP by interaction with integrins and thrombospondin; and c) rupture of the noncovalent bonds between LAP and mature TGF-β1 (Annes et al., 2003. J Cell Sci. 116, 217-224). Once released, TGFβs can bind to their specific cell surface receptor (three components, type I, type II and type III) to induce signalling through downstream effectors (e.g. Smad proteins).

Since many mechanisms may stimulate cells to activate Latent TGFβ, the mechanism and timing of activation of Latent TGFβs appears to be specific for each cell and tissue type. In fact, the existence of different genes encoding functionally similar proteins, yet controlled by differentially regulated promoters (Roberts et al., 1991. Ciba Found. Symp., 157, 7-28), provides an important mechanism to ensure tissue-specific and spatio-temporal expression patterns of the different TGFβs isoforms, resulting in proper cell and tissue behaviour (Piek et al., 1999. FASEB J., 13, 2105-2124). Accordingly, the present invention can be used to influence specific cell types in specific tissues by immobilizing the appropriate latent growth factor or combinations of latent growth factors.

In mammals there are three isoforms of the TGFβ, namely, TGFβ1, -β2 and -β3 (Li et al., 2006. Annu Rev. Immunol., 24, 99-146) involved in a multitude of in vivo functions (for review see Katrien et al., 2005. Endocrine Reviews, 26, 6, 743-774). Almost all cells have receptors for TGF-βs and produce at least one of these isoforms.

TGFβ1 is a ubiquitous (platelets and bone contain the largest amounts) and multifunctional growth factor that is implicated in many cell processes (migration, proliferation, differentiation, survival, production of ECM) (Moses H L, Serra R., 1996. Curr Opin Genet Dev., 6, 581-586) influencing processes such as embryogenesis, angiogenesis, vascuologenesis, inflammation (mainly anti-inflammatory effect depending on the context), immunoregulation (usually immunosupressor), wound healing and maintenance of tissue homeostasis during life (Gorelik & Flavell, 2002. Nat Rev Immunol. 2, 46-53; ten Dijke & Arthur, 2007. Nat. Rev Mol Cell Biol., 8, 857-869; Roberts A B, 1998. Miner Electrolyte Metab., 24, 111-119). In skeletal tissue, TGFβ1 plays a major role in development and maintenance, affecting both cartilage (Mehlhom et al., 2007. Cell Prolif., 40, 6, 809-23) and bone (Ripamonti et al., 2006. J Anat., 209, 4, 447-68) metabolism. It has a crucial role retaining the balance between the dynamic processes of bone resorption and bone formation. Bone formation is promoted by TGFβ1 through chemotactic attraction of osteoblasts, enhancement of osteoblast proliferation and the early stages of differentiation with production of ECM proteins, stimulation of type II collagen expression and proteoglycan synthesis by chondrocyte precursor cells and suppression of hematopoietic precursor cell proliferation. Growth plate chondrocytes are particularly sensitive to TGF-β1, responding to levels that are 10-fold less than those that modulate osteoblast-like cells under similar conditions (Dallas et al., 1994. J. Biol. Chem., 269, 6815-6822). At later stages of endochondral differentiation, TGF-β1 suppresses chondrocyte hypertrophy and matrix calcification (Ballock et al., 1993, Dev Biol., 158, 414-429, 1993). Among other growth factors (IGFs, FGFs, PDGFs, and EGFs), TGFβs are the most potent inducers of chondrogenesis and enhancement of cartilage ECM synthesis in chondrocytes (Vunjak-Novakovic et al., 2005. Orthod Craniofac Res., 8, 209-218). As exemplified below, the latent form of TGFβ1 was immobilized in nanofibrous scaffolds and human chondrocytes were used to demonstrate proof of concept in vitro.

Additionally, besides bone and cartilage, TGF-β1 is involved directly or indirectly in the regulation of other cell types such as hepatocytes (Chia et al., 2005. Biotechnol Bioeng., 5, 89, 5, 565-73), vascular endothelial cells (Sales et al., 2006. Circulation, 114(1 Suppl):I193-9), cardiac fibroblasts (Caraci et al., 2008. Pharmacol Res., 57, 4, 274-82; Lim et al., 2007. Mol Cells, 31, 24, 3, 431-6), lamina cribrosa cells from the human optic nerve (Kirwan et al., 2004. J Glaucoma. 13, 4, 327-34), retinal epithelial cells (Uchida et al., 2008. Curr Eye Res., 33, 2, 199-203), renal mesangial cells (Huang et al., 2008. Am J Physiol Renal Physiol., March 26—epub), extraocular muscle cells (Anderson et al. 2008. Invest Ophthalmol Vis Sci., 49, 1, 221-9), mouse mesencephalic progenitors (Roussa et al., 2006. Stem Cells, 24, 9, 2120-9), intestinal epithelial cells (Kurokowa et. Al., 1987. Biochem Biophys Res Commun., 142, 775-782), bronchial epithelial cells (Masui et al., 1986. PNAS, 83, 2483-42) controlling or influencing the differentiation, transdifferentiation, migration, ECM production or avoiding matrix degradation amongst other cell behaviours. Accordingly, based on the plethora and potential effects of TGFβs on different tissues, the present invention can be used to target several diseases in different types of tissues, such as in bone (osteogenesis), cartilage (chondrogenesis), cardiac disease (e.g. aortic valves modified with LTGF), ophthalmologic disease (e.g. transplantable retinal epithelial prepared from nanofiber sheets modified with LTGF) or renal disease, but not limited to these. For application in the treatment of renal disease it is of note that latent TGF-β1 has been reported to protect against renal inflammation in Crescentic Glomerulonephritis (Huang et al., 2008. J Am. Soc. Nephrol., 19, 2, 233-42).

The TGFβ isoforms are thought to have similar functions. A few studies report the potential effect of TGFβ2 and TGFβ3 in cell function. For example, TGF-β2 has been used successfully to treat one of most frequent and severe side-effects of chemotherapy in childhood-cancer patients (Mucositis) (Koning et al., 2007, Pediatr. Blood Cancer, 48, 5, 532-9) and locally applied in orthopaedic implants to increase the peri-implant bone volume and bone-implant contact (Ranieri et al., 2005. Bone, 37, 1, 55-62). TGF-β3 has been described to protect against an inflammatory demyelinating disease of the CNS, the Experimental Autoimmune Encephalomyelitis (EAE) (Agata et al., 2003. Cytokine, 25, 2, 45-51). Because all three TGF-β isoforms are detected in many tissues, the present invention can be used to immobilize latent TGF-β1, latent TGF-β2 and latent TGF-β3, or any combination thereof.

Additionally, latent TGF-β1, latent TGF-β2 and latent TGF-β3 can be immobilized in different proportions and combinations to achieve different cell responses, such as recreating the precise role of the three isoforms in bone (effect on mineralization) and cartilage, as well as in other tissues.

As stated above, the present invention can be applied for the immobilization of other known or unknown latent forms of any growth factor or cytokine. Besides TGFβ1, TGFβ2 and TGFβ3, other cytokines that belong to the TGFβ superfamily are produced in latent forms, such as activins and inhibins, bone morphogenetic proteins (BMPs) such as BMP7, (Gregory et al., 2005. J Biol Chem. 29, 280, 30, 27970-80) growth differentiation factors (GDFs) (Gaoxiang et al., 2005. Mol. Cel. Biol., 25, 14, 5846-5858). The latency of the growth factor may be provided by association of the growth factor with a latency associated protein, such as the TGFβ LAP. Growth factors can be provided in association with latency associated proteins by methods known in the art. Accordingly the present invention can be applied to these or combinations thereof.

Therefore, the invention can be used to prepare biomaterials presenting one or more latent growth factors to be used as effective bioactive scaffolds.

The present invention can be used with other alternative bioactive molecules obtained as recombinant growth factors associated with a fusion protein comprising a latency associated peptide (LAP) and a specific proteolytic cleavage site (e.g. which can be cleaved by MMPs), in order to provide latency to those bioactive molecules. Thus, the latent form of the growth factor can comprise a growth factor associated with a latency associated peptide.

The latent form of the growth factor can comprise the active form fused together with one or more latency associated peptides, as described above in the example of TGFβ. Alternatively, the latency associated peptide can be associated with the active form of the growth factor by any covalent or non-covalent interactions. Any growth factor which is provided in a form wherein the activity of the growth factor is repressed until activation by a cellular signal can be used in the present invention.

The latent form of the growth factor can be immobilised on the biomaterial by any suitable means. The latent form of the growth factor can be immobilised directly to the surface of the biomaterial, for example, by covalent linkage or by means of non-covalent interactions, such as ionic bonds, hydrophobic interactions, hydrogen bonds, Van der Waals forces, dipole-dipole bonds, and π-π interactions. The particular means of immobilisation will depend on the type of biomaterial used.

Alternatively, the latent form of the growth factor can be immobilised to the biomaterial via one or more intermediate molecules. For example, an antibody specific for the latent form of the growth factor can be immobilised to the biomaterial surface by any of the immobilisation methods described herein, and the latent form of the growth factor can be bound to the antibody. The antibody may be polyclonal, monoclonal or recombinant. In addition to whole antibodies, fragments or derivatives thereof which are capable of binding to the latent form of the growth factor can also be used. Thus the intermediate molecule may be an antibody fragment or a synthetic construct capable of binding the latent form of the growth factor. Examples of antibody fragments and synthetic constructs are given by Dougall and co-workers (Dougall et al., Tibtec, 12: 372-379, 1994). Antibody fragments include, for example, Fab, F(ab′)₂ and Fv fragments. Fab fragments are discussed in Roitt et al, Immunology second edition (1989), Churchill Livingstone, London. Fv fragments can be modified to produce a synthetic construct known as a single chain Fv (scFv) molecule. This includes a peptide linker covalently joining Vₕ and V₁ regions, which contributes to the stability of the molecule. Other synthetic constructs that can be used include Complementarity Determining Regions (CDR) peptides. These are synthetic peptides comprising antigen-binding determinants. Other synthetic constructs which can be used include chimaeric molecules, which include variable regions from a non-human mammal (such as a mouse or rat) and human constant regions. In addition, synthetic constructs include humanised (or primatised) antibodies or derivatives thereof, in which the antibody is human except for the complementarity-determining regions, which are taken from a non-human mammal. Ways of producing chimaeric/humanised antibodies are discussed for example by Morrison et al in PNAS (81: 6851-6855, 1984) and by Takeda et al in Nature (314: 452-454, 1985). Peptide mimetics may also be used. These molecules are usually conformationally restricted organic rings that mimic the structure of a CDR loop and that include antigen-interactive side chains.

Commercially available antibodies or fragments thereof, which are known to specifically bind to the latent form of the growth factor, may be used. Alternatively, antibodies may be raised to the latent form of the growth factor by methods known in the art. Techniques for producing monoclonal and polyclonal antibodies that bind to peptide/protein are now well developed (Roitt et al., Roitt's Essential Immunology, 2006). Polyclonal antibodies can be raised by stimulating their production in a suitable animal host (e.g. mouse, rat, guinea pig, rabbit, sheep, goat, monkey, horse, pig) after immunization with the appropriate immunoconjugate. Monoclonal antibodies can be produced after immunization with the appropriate immunoconjugate, by fusing spleen lymphocytes with myeloma cells (e.g. P3-X63/Ag 8.653) and further screening the fused cells for the presence of antibodies that recognize the latent form of the growth factor (Kohler & Milstein, Nature, 1975, 256, 52-55). Selected hybridomas can be cloned and expanded and the antibody purified by affinity-chromatography (Nowinski et al., Virology 1979, 93, 111-126).

The one or more intermediate molecule may be immobilised to the biomaterial by any suitable means known in the art.

The latent form of the growth factor can be immobilised on the biomaterial via specific sites on the latent growth factor molecule (or intermediate molecule) to ensure that the latent growth factor is provided in a particular orientation. The latent growth factor may be more effective when immobilised on the biomaterial in a particular orientation. Alternatively, the latent form of the growth factor can be immobilised on the biomaterial via non-specific sites on the latent growth factor molecule (or intermediate molecule) (i.e., randomly). Greater quantities of the latent growth factor may be immobilised on the biomaterial using methods that involve the random immobilisation of the latent growth factor to the biomaterial.

The immobilization of the latent TGFβ or any other latent growth factor can be performed using standard conjugation chemistry (or by any other means known in the art) depending on the functional groups available on the biomaterial/scaffold. For example, the biomaterial may be modified using a suitable plasma technique (one example of which is described in more detail in the example below) to introduce amine, carboxyl, hydroxyl, vinyl and other reactive groups onto the surface thereof. In the case of amine groups, Sulfo-SMCC (Sulfosuccinimidyl-4-(N-maleimidomethyl)cyclohexane-1-carboxylate) may be used to form covalent bonds with amine groups on the biomaterial surface and also with thiol groups present on the latent form of a growth factor (e.g. TGFβ). Thus, the latent form of TGFβ may be immobilised to the biomaterial via terminal thiol groups present on the latent form of TGFβ. The terminal thiol groups may be covalently or non-covalently bound directly to the biomaterial or to functional groups applied to the biomaterial. Alternatively, the terminal thiol groups may be covalently or non-covalently bound to the biomaterial indirectly, via an intermediate molecule.

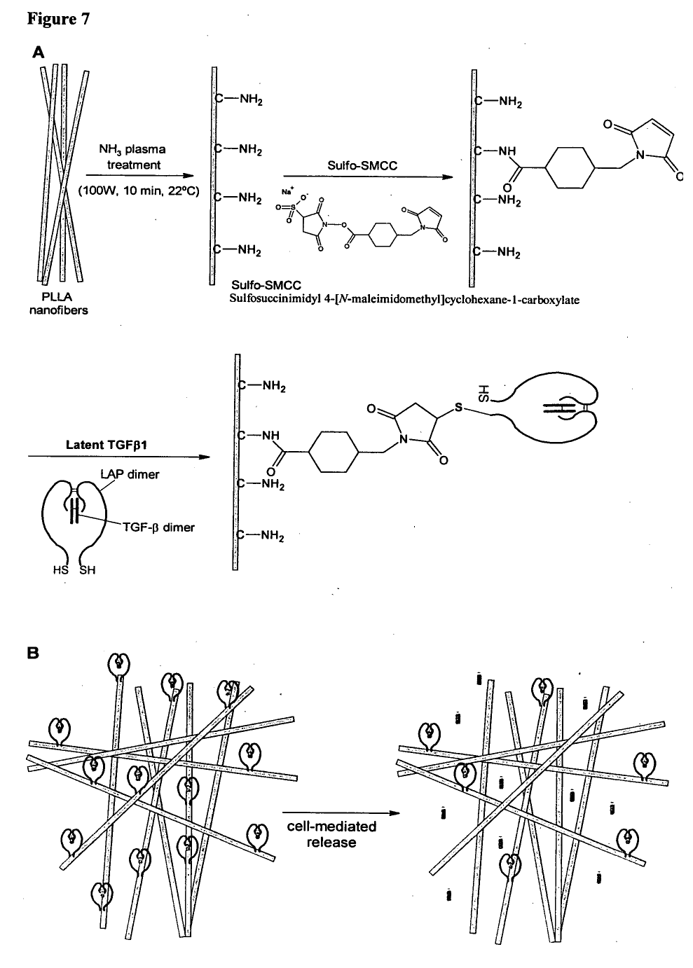

Preferentially, when the growth factor is TGFβ, the biomaterial may be modified with amine groups and further functionalized with maleimide groups which can specifically react with the terminal thiol groups on the SLC molecule (see FIG. 7). This way the SLC will remain orientated once immobilized at the material surface mimicking the presented conformation under normal in vivo ECM physiological conditions before activation. Thus, the latent growth factor may be more effective in vivo when immobilised on the biomaterial in a particular orientation. Chemical coupling can be performed by using for example Sulfo-SMCC (see example below) but other similar reagents can be used (e.g. Sulfo-EMCS ([N-e-Maleimidocaproyloxy]sulfosuccinimide ester, GMBS (N-[g-Maleimidobutyryloxy]succinimide ester, and others known in the art). However, other chemical conjugation strategies can be, followed including for example molecules that contain a NHS terminal and a pyridyl disulfide terminal group (e.g. Sulfo-LC-SPDP, Sulfosuccinimidyl 6-(3′-[2-pyridyldithio]-propionamido) hexanoate; Sulfo-LC-SMPT (4-Sulfosuccinimidyl-6-methyl-a-(2-pyridyldithio)toluamido]hexanoate; and any other appropriate coupling reagents commercially available) that can further interchange with free SH groups on SLC.

Alternatively, SLC can be immobilized by pre-functionalising the biomaterial with specific antibodies to the N-terminal of the SLC.

Furthermore, the SLC could be immobilized using specific peptides from the latent TGFβ binding proteins from LTBP1, LTBP2 or LTBP3 or mixtures thereof.

Alternatively, the SLC may be randomly immobilized throughout the scaffold using functional groups other than just thiols of the SLC terminal.

Alternatively, as stated above, simple strong physical adsorption to the scaffold involving hydrophobic interactions or others are also envisioned.

Additionally, the immobilization of specific latent forms of growth factors can be directed to specific locations within the scaffold promoting different cell responses or triggering activation by/of different cell types.

Alternatively, different materials, containing different latent forms or different surface densities of the latent forms can be mixed to achieve better cell responses.

Thus, in another aspect, the invention provides a method of producing the biomaterial of the invention, comprising immobilising the latent form of the growth factor on the biomaterial. The method may comprise the steps:
modifying the biomaterial to provide functional groups which are capable of binding to functional groups on the latent form of the growth factor; and
contacting the modified biomaterial with the latent form of the growth factor under conditions which allow the latent form of the growth factor to bind to the functional groups on the biomaterial.

The functional groups may be any compound capable of achieving a specific binding interaction with the latent form of the growth factor. The method may include any of the immobilisation techniques described herein.

The biomaterial having a latent form of a growth factor immobilised thereon may further comprise one or more cells thereon. The cells may be immobilised or seeded on the biomaterial. For example, the cells may be prepared in an aqueous suspension, which then may be added to the biomaterial. Thus, the cells may form strong or weak attachments to the biomaterial, and may be either retained in a fixed position on the biomaterial or may be capable of moving relative to the biomaterial surface. The cells may be progenitor cells. The progenitor cells may be embryonic stem cells, foetal stem cells, umbilical cord or placental stem cells, or adult stem cells (such as mesenchymal or haematopoietic cells), or other stem cells. The cells may be derived from bone marrow, or adipose tissue (but not limited to these) and may be other primary cells. For example, the cells may be chondrocytes (such as nasal or articular chondrocytes) or human periosteum-derived cells. The particular cell will depend on the intended use of the biomaterial. The cells can be derived from any animal species into which the biomaterial is intended to be implanted. Preferably, the cells are human cells.

Also, the present invention can be combined with the concomitant use of other immobilized or free in solution forms of the growth factors, since many of the TGFβ effects are enhanced by the presence of the other cytokines (e.g IL-2 for the differentiation of T-cells).

Any other molecules that co-regulate the role of the TGFβ superfamily can be used as well, such as hormones (Dexamethasone), vitamins (Vitamine D) and others known in the art.

Additionally, the biomaterial or scaffold may contain a mixture of materials (e.g. different fibres) modified for example with specific osteoinductive, chondroinductive, angiogenic peptides, but not limited to these, together with single or multiple growth factor modified materials.

The activation of the immobilized latent growth factor can be co-controlled by the addition of specific activation inhibitors such as peptides.

Accordingly, th present invention describes a new strategy to influence and direct cell behaviour taking advantage of the latent form of a growth factor (exemplified as TGF-β1 stored inside the Small Latent TGF-β1). The procedure involves the immobilization/accumulation of the latent growth factor form at the surface of biomaterials (e.g. nanofibers) as a pool of bioactive molecules ready to be used, but still in its inactive form. The associated peptide or LAP, confers latency to the mature peptide of the TGF-β1 isoform, shielding the epitopes that interact with the receptor, preventing immediate downstream signalling. Additionally, LAP acts as a stabilizer, protecting TGF-β1 from degradation and inactivation and possesses important residues for interaction with other molecules. When latent form of TGFβ1 is presented at the surface of scaffolds, cells can interact with it and mediate the activation of the reservoir of latent TGF-β1 according to cell demand, releasing totally or partially (a percentage remains resident of the scaffold) the active form of the growth factor locally. The biological activity of the soluble growth factor (GF) will then be available for cell receptor interaction, triggering intracellular cascade signalling. The invention provides a closer approximation to the in vivo situation than other controlled growth factor delivery attempts. In bone, the cells efficiently secrete large amount of the SLC (indeed this is the predominant form of the growth factor in bone) (Bonewald et al., 1999. Mol. Endocrinol., 5, 741-751). The presence of other growth factors in the environment, the cell differentiation stage and the environment as such will determine the activation or not of the available latent form. This way the cell itself regulates the activation and release of the growth factor at the appropriate time and concentration, determining the exact outcome of the growth factor on cell activity. As stated above, the invention covers other latent forms of growth factors as well as latent fusion proteins.

In a further aspect, the invention provides the use of the biomaterial of the invention in vitro. The in vitro methods of the invention can be used to develop initial cell differentiation and/or growth before implantation of the biomaterial into a patient. In addition, the invention can be used to generate bioactive materials as cell supports for in vitro cell culture and differentiation (such as for bone or cartilage growth and/or differentiation). Furthermore, the invention can be used in combination with bioreactor systems (e.g. hydrodynamic bioreactors) to enhance the growth of cell constructs either by nutrient supply and/or mechanical stimulation for example.

The present invention can also be used to study mechanotransduction signaling pathways using latent growth factors (such as TGFβ) immobilized to biomaterials.

In another aspect, the invention provides the use of a biomaterial of the invention in medicine. For example, the invention provides the use of a biomaterial of the invention in tissue regeneration or repair. For example, a biomaterial having a latent form of TGFβ immobilised thereon can be used in the repair or regeneration of bone and/or cartilage.

In another aspect, the invention provides a method of treating tissue damage in a patient, comprising implanting the biomaterial of the invention into the patient.

The biomaterials of the invention can be used in the treatment of an animal such as a mammal, and preferably a human. Other animals which can be treated using the biomaterials of the invention include domesticated animals such as dogs, cats, rabbits, horses, guinea pigs, etc. and cattle such as sheep, cows, pigs, goats, chickens, etc, and others.

The biomaterials of the invention can be used to repair (i.e. to induce regeneration and growth) of tissue damaged by disease or injury.

When the growth factor is TGFβ, the biomaterials of the invention can be used in the treatment of several diseases in several tissue types, such As such as in bone (osteogenesis), cartilage (chondrogenesis), cardiac disease (e.g. aortic valves modified with LTGF), ophthalmologic disease (e.g. transplantable retinal epithelial prepared from nanofiber sheets modified with LTGF) or renal disease, but not limited to these.

Because cells from the immune system (e.g. macrophages) can release the bioactive TGFβ from its latent form, the present invention could be used to exert a powerful anti-inflammatory effect in certain specific conditions, depending on the context, because TGF-β may be underproduced in some autoimmune diseases, but it is overproduced in many pathological conditions like pulmonary fibrosis, Crohn's disease, among others.

Another important area of application is in wound healing repair because TGFβ stimulates this process in collaboration with other growth factors (Hyytiainen et al., 2004, Crit Rev Clin Lab. Sci., 4, 233-264). Accordingly, the present invention is useful in cardiac remodelling after ischemic injury, also because there is recent evidence that TGF-β1 can protect cardiomyocytes from ischemic injury (Bujak & Frangogiannis, 2007. Cardiovasc. Res., 74, 184-195).

Preferred features of each aspect of the invention are as for each of the other aspects mutatis mutandis. The prior art documents mentioned herein are incorporated to the fullest extent permitted by law.

Particular embodiments of the invention will now be discussed with reference to the accompanying drawings in which:

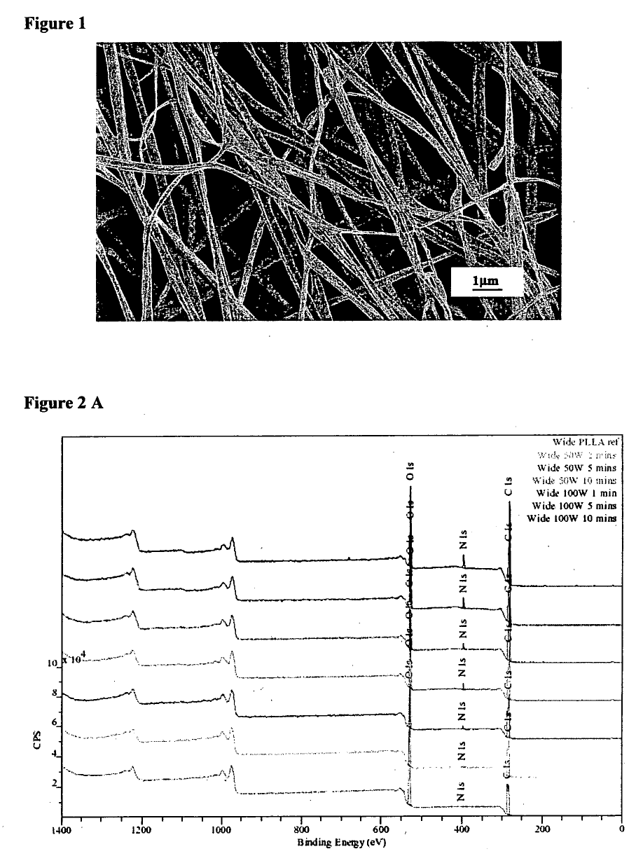

FIG. 1—Representative SEM photograph showing the morphology of PLLA fibres produced by electrospinning.

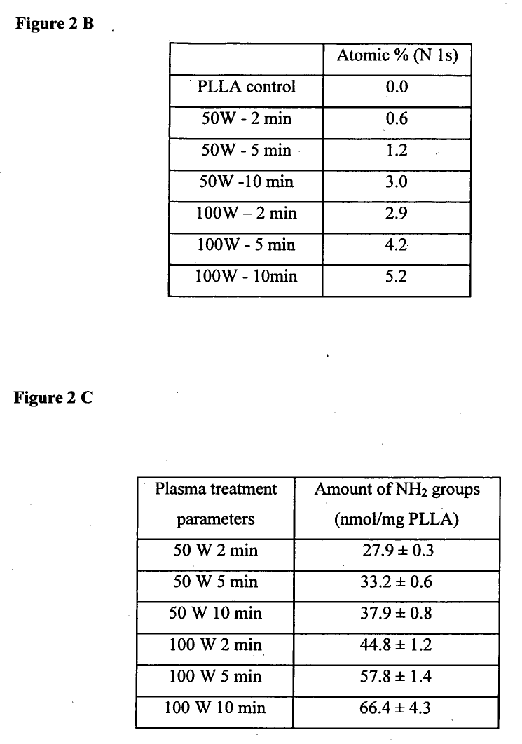

FIG. 2—A—X-ray Photoelectron Spectra of untreated and ammonia plasma treated PLLA fibres using different exposure power (W) and time (min);

B—Calculated amount of atomic percentage of N 1 s on the surface of scaffolds;

C—Calculated amount of NH₂ groups (nmol/mg PLLA) on the surface of scaffolds.

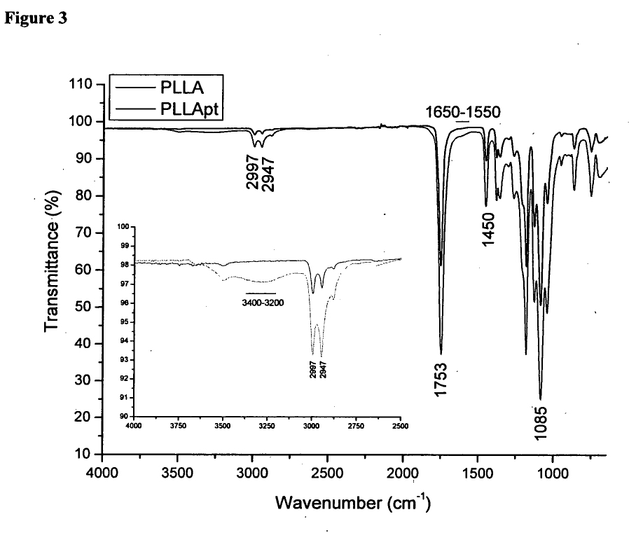

FIG. 3—ATR-FTIR spectroscopy of untreated PLLA and plasma treated PLLA.

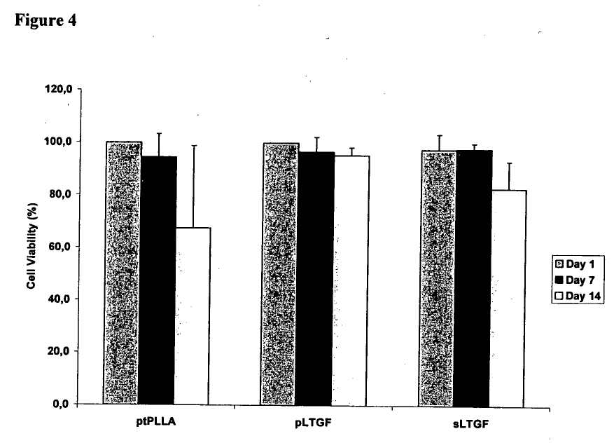

FIG. 4—Cell viability of human articular Chondrocytes based on the Live/Dead assay. pLTGF=LTGF randomly linked to plasma treated PLLA (ptPLLA); sLTGF=LTGF oriented immobilized on ptPLLA modified with Sulfo-SMCC.

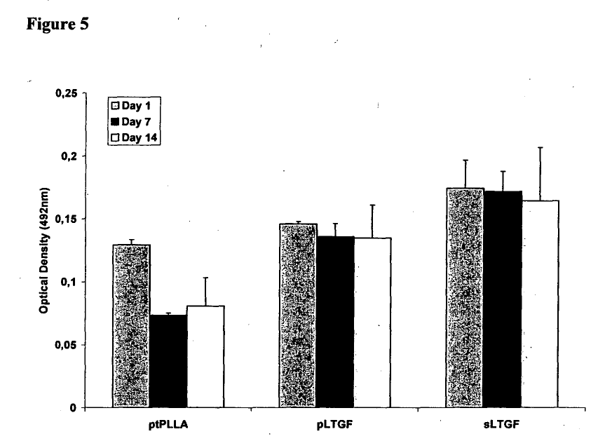

FIG. 5—Evaluation of cell viability/proliferation of human articular chondrocytes on the scaffolds using the MTS assay. The results represent a mean±SD of triplicates cultures from two experiments. pLTGF=LTGF randomly linked to plasma treated (ptPLLA); sLTGF=LTGF oriented immobilized on ptPLLA modified with Sulfo-SMCC

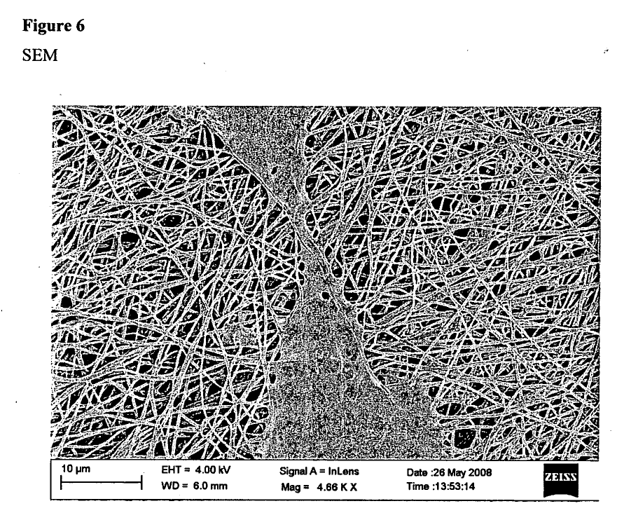

FIG. 6—SEM photographs of human articular chondrocytes over plasma treated PLLA modified with Sulfo-SMCC and LTGF (sLTGF) after cultured for 7 days.

FIG. 7—Illustrates a possible method for production of modified PLLA fibres to which the latent growth factor is covalently linked. PLLA electrospun fibres were subjected to NH₃ plasma treatment and further functionalised using the heterobifunctional Sulfo-SMCC. The latent growth factor complex was further covalently immobilized through its thiol groups.

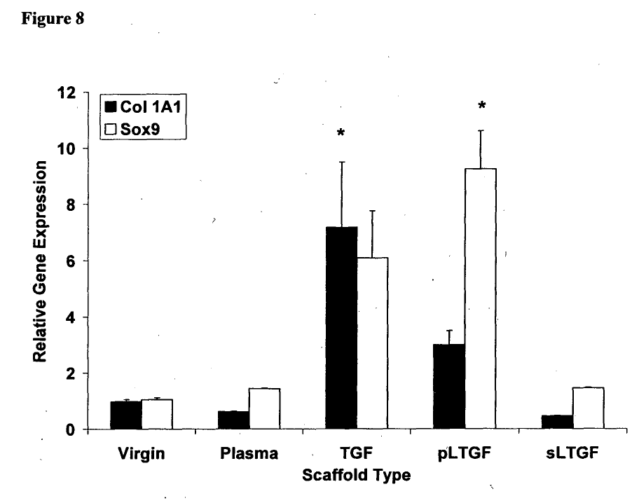

FIG. 8—Real-time RT-PCR data showing mRNA expression profiles of primary human nasal chondrocytes cultured on various scaffold types for 14 days. Expression levels of selected chondrogeneic (Sox9) and dedifferentiation (Col1A1) markers were normalised to the 18S housekeeping gene and day 0 expression levels. Average change±standard deviation of repeated experiments (n=3) are presented. Col1A1 of TGF group and Sox9 of pLTGF group were significantly higher when compared to the rest of the groups of the respective genes (*p<0.05). (Virgin=untreated electrospun scaffolds; Plasma=plasma treated electrospun scaffolds; TGF=plasma treated electrospun scaffolds with media supplementation of 10 ng/mL active human recombinant TGF-β1 for up to 7 days; pLTGF=LTGF randomly linked to plasma treated PLLA (ptPLLA); sLTGF=LTGF oriented immobilized on ptPLLA modified with Sulfo-SMCC)

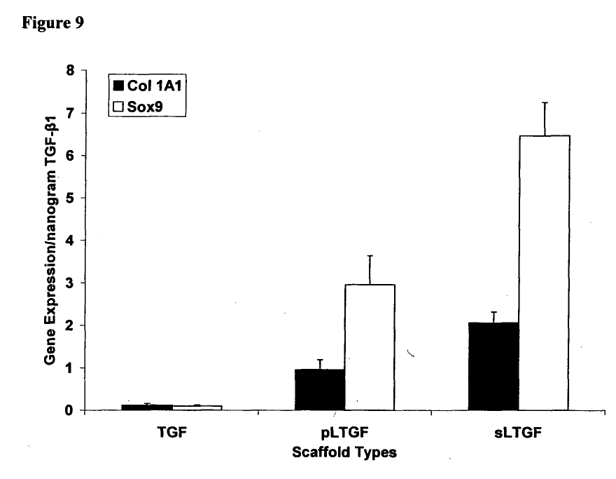

FIG. 9—Efficacy in induction of human nasal chondrogeneic differentiation per nanogram of TGF-β1. Relative gene expression results of the respective groups were divided by the amount of TGF-β1 present or supplemented during the course of the experiment. Means and standard deviations are presented.

### EXAMPLE

Preparation and Modification of Poly-L-Lactic Acid (PLLA) Fibres with Recombinant Latent TGFβ1 and Evaluation of Seeded Human Chondrocytes Activity.

Materials and Methods

PLLA fibres were prepared in 10×15 cm sheets (FIG. 1) with ˜4 mm thickness using a home made electrospinning system. The nonwoven scaffold was spun from a 3 wt % PLLA (Mw˜300 KDa, Purac) solution in dichloromethane/dimethylformamide (70:30, w:w) with an applied voltage of 10 kV and a rate delivery of 1 mL/min. The fibres were collected on a rotating aluminium mandrel.

Plasma Treatment

Since reactive functional groups, easily modified, are absent in the backbone of PLLA, it is difficult to modify the surface by common chemical methods. The utilization of plasma technique has been widely used to introduce desired chemical groups onto the surface of materials (Flavia & D'Agostino, Surf Coat Technol., 98, 1102-06, 1998) including PLLA. Thus, in order to introduce amine functional groups on the PLLA electrospun fibres, samples of PLLA were modified using a conventional NH₃ plasma treatment (Yang et al., J Biom. Mater Res., 67A, 1139-47, 2003). Samples were previously cut in to 1×1 cm squares and evenly distributed over a sterile glass Petri dish. Samples were sterilized with UV for 30 min. and further submerged in ethanol for 1 h. Once dry, samples were placed in the plasma chamber and the pressure reduced until —10 Pa before filling the NH₃ gas. After the pressure of the chamber had stabilized, a glow discharge plasma was created by controlling the electrical power at a radio frequency of 13.56 MHz for a predetermined time (2.5 min, 5 min and 10 min; half of this time in each side of the sample). The plasma-treated samples were further exposed to ammonia gas for another 10 min before the sample was taken out (according with Yang et al., 2002. Biomaterials, 2607-2614). The plasma treated samples were immediately enclosed within a sterile petri dish and taken to the cell culture cabinet for further modification.

Scanning Electron Microscopy (SEM) Analysis

SEM analysis was used to monitor cell attachment and morphology. At different time points scaffolds containing cells were fixed with gluteraldehyde 2.5% in cacodylate buffer pH 7.4 for 2 hours at 4° C., dehydrated through a series of increasing concentrations of ethanol and finally dried by immersing in 100% hexamethyldisilazane (HMDS). After drying the samples were mounted on an aluminium stub, sputter coated with chromium and viewed with a scanning electron microscope (FEGSEM, Leo 1525) using an accelerating voltage of 5 KV.

Surface Chemistry Analysis Using X-Ray Photoelectron Spectroscopy (XPS) and Ninhydrin Assay

To analyse the successful modification of the surface by NH₃ plasma treatment, samples were analysed before and after modification by X-ray photoelectron spectroscopy (XPS) on a Kratos-Axis ULTRA DLD XPS instrument, operated at 10 mA emission and 10 KV anode potential (100 W) under vacuum (<3×10⁻⁹ Ton). Data was analysed using CASAXPS software with Kratos sensitivity factors to determine atomic percentage (%) values from the peak areas. The high resolution spectra C1s was deconvoluted and curve-fitted to analyse the chemical bonding state using appropriate controls.

The ninhydrin assay was used to quantitatively detect the amount of amine groups on the ammonia plasma surface modified PLLA scaffolds. The assay was performed as previously described (Zhu et al., 2002, Biomacromolecules, 11, 3, 1312-1319; Zhu et al., 2004, Tissue Eng., 1, 10, 53-61).

ATR-FTIR Analysis

The ATR-FTIR spectra of untreated and NH₃ plasma-treated PLLA were obtained with a Perkin Elmer 2000 FTIR in the region from 650 to 4000 cm⁻¹, with resolution of 4 cm⁻¹ and 16 scans per sample.

Recombinant Latent TGFβ Immobilization

A schematic illustration of the immobilization of the LTGFβ1 onto the PLLA scaffold is shown in FIG. 7. For the oriented covalent immobilization of the SLC through its two free tiols on the N-terminal of LAP, amine groups on the plasma treated PLLA fibers were modified with Sulfo-SMCC (Sulfosuccinimidyl-4-(N-maleimidomethyl)cyclohexane-1-carboxylate). This heterobifunctional cross-linker is water-soluble and contains an amine-reactive N-hydroxysuccinimide (NHS ester) and a sulfhydryl-reactive maleimide group. The NHS ester will react with the primary amine on the PLLA fibers at pH 7 to form stable amide bonds whereas the maleimide group will react with the free sulfhydryl groups at SLC at pH 6.5-7.5 to form stable thioether bonds. The maleimide groups of Sulfo-SMCC and SMCC are unusually stable up to pH 7.5 because of the cyclohexane bridge in the spacer arm. Because it contains the hydrophilic sulfonyl moiety, Sulfo-SMCC is soluble in water, thus avoiding the use of organic solvents that may perturb the fibre PLLA structure. Freshly ammonia plasma treated PLLA samples were submerged for 30 minutes in sterile PBS and after that submerged in a 3 mg/ml Sulfo-SMCC freshly prepared solution in PBS and left to react for 2 h under agitation (orbital shacker) inside the sterile cell culture safety cabinet. The supernatant was discarded and the scaffold rinsed three times with sterile PBS. The functionalized scaffolds were then submerged in a solution of recombinant latent TGFβ1 (0.75 μg/mL) (freshly prepared) in sterile PBS for 2 h. The supernatant was removed, the scaffolds rinsed three times with sterile PBS and placed individually in cell culture plates for cell seeding.

For the random immobilization of the recombinant latent TGFβ1 onto the PLLA, freshly prepared NH₃ plasma-treated PLLA samples were immediately submerged in a 0.75 μg/mL SLC (freshly prepared) in PBS for 2 h. The supernatant was removed, the scaffold rinsed three times with sterile PBS and placed individually in cell culture plate for cell seeding.

Quantification of Latent TGF-β1 on Scaffolds Using an Immunoassay

The measurement of immobilized LTGF-β1 was performed using a modified procedure described previously (Pedrozo et al., 1998, J Cell Physiol., 177, 343-354). Modified scaffolds and appropriate controls were initially digested with 0.3 U/mL of plasmin (Sigma-Aldrich, UK) in DMEM for 3 hours at 37° C. to release the LTGF-β1 from the scaffold. The reaction was stopped by the addition of aprotinin (Sigma-Aldrich, UK) to a final concentration of 5 μg/mL. The supernatant was collected and the immunoreactive TGF-β1 was liberated from the LTGF-β1 complex by acidification of the supernatant with 20 μL of 1 M HCl to every 100 μL of supernatant, at room temperature for 10 minutes. The acidified supernatant was neutralized with 1.2 N NaOH/0.5 M HEPES to pH ˜7.3 and immediately used for quantification of TGF-β1 using a Quantikine Human TGF-β1 Immunoassay (R&D Systems, UK) following manufacturer's protocol.

Cell Culture and Seeding

In this example we used chondrocytes isolated from human nasal septal cartilage from a 45 year old healthy patient (with full ethical consent) and adult human articular chondrocytes obtained from Lonza (Lonza Walkersville, Md.). Cells were culture panded in tissue culture plastic (TCP) flasks in basal chondrocyte growth medium (bCGM), which was phenol-red free DMEM (Gibco 41966) supplemented with 10% (v:v) FBS, 2 mM L-glutamine (GIBCO), 0.1 mM nonessential amino acids (GIBCO), 100 U/ml penicillin, 100 μg/mL streptomycin, 50 μg/mL ascorbic acid-2-phosphate according to a previous report. All cell cultures were maintained at 37° C. in an incubator with 95% air and 5% CO₂. The cultures were replenished with fresh medium at 37° C. every 3 days.

For scaffold seeding, cultured cells were trypsinized, harvested, counted and ressuspended in a small volume of bCGM before being evenly seeded drop-wise onto scaffolds previously conditioned in DMEM for 30 minutes at 37° C. Constructs were incubated for 4 hours at 37° C. in the cell incubator to allow cells to diffuse into and attach to scaffolds before fresh serum-free media was added (see below). Scaffolds were seeded at 9000 cells/cm² onto 1.5×1.5 cm² scaffolds. For gene expression studies samples were seeded at 3.5×10⁴ cells/cm² on 4×4 cm² scaffolds. Pellets containing 5×10⁵ cells were snap-frozen and used as day-0 specimen for gene expression analysis.

To evaluate the effects of the scaffold-immobilized LTGFβ1 on cell cultured chondrocytes a serum-free chemically defined differentiation medium was used throughout the experiment. The serum-free media consisted of bCGM without FBS supplemented with ITS+premix (BD Bioscience) (6.25 μg/ml insulin, 6.25 μg/ml transferrin, 6.25 μg/ml selenium, 1.25 mg/ml bovine serum albumin, 5.33 μg/ml linoleic acid; Sigma) and 100 nM dexamethasone (Li et al., 2005. Biomaterials, 26, 599-609). The media was replaced every 3 days.

Cell Viability

The viability of chondrocytes cultured in the scaffolds was examined by a Live/Dead assay (Molecular Probes, Eugene, Oreg.). Briefly, scaffolds seeded with chondrocytes were washed with PBS, protected from light and incubated in 2 μM calcein AM (staining live cells) and 4 μM EthD-1 (staining dead cells) in PBS for 30-45 min at room temperature. Then, each sample was washed with PBS before evaluation using an inverted fluorescence microscope equipped with a digital camera and appropriate software for image analysis. Images were taken in different areas and in both sides in order to evaluate cell distribution. The number of viable cells (green) and dead (red) cells was counted and cell viability expressed as number of viable cells (green) per total number of cells (green+red).

Cell Proliferation

Cell proliferation on scaffolds was assessed by measuring the cell metabolic activity using the CellTiter 96Aqueous One Solution Cell Proliferation Assay (Promega) following the manufacturer's instructions. Briefly, scaffolds seeded with cells were transferred to new well-plates and rinsed gently with sterile PBS. Samples were than incubated with 400 μL phenol free medium plus 80 ∞L. CellTiter 96 reagent and left to react for 4 h at 37° C. in a humidified 5% CO₂ environment. Optical density of the supernatant was measured at 490 nm using a microplate reader (Anthos Biotech). A sample cultured under the same conditions in the absence of cells was used as a blank. The results represent the mean values of two individual experiments, each in triplicate.

Real-Time Reverse Transcriptase Polymerase Chain Reaction (RT-PCR)

Total RNA was extracted from chondrocytes by the addition of 10 μL, β-mercaptoethanol in 1 mL RLT-Buffer (QIAGEN, UK). Total RNA was isolated using the RNeasy mini kit, treated with RNase-free DNase 1 (both QIAGEN), according to the manufacturer's protocol and quantified using the ND-1000 UV-Vis Spectrophotometer (Nanodrop®, USA). One microgram of total. RNA was reverse transcribed into cDNA and a mastermix prepared for each reaction containing: 10 μL Taqman™ universal mastermix (Applied Biosystems, USA), 7 μL 0.1% (v/v) diethylpyrocarbonate (DSPC) water (Invitrogen Ltd, UK), 2 μL extracted cDNA and 1 μL Taqman probe (Applied Biosystems, USA). TaqMan® Gene Expression Assay kits were used to amplify cartilage-related genes including Collagen type-1 (Col1A1-1, NM_{—}000088), Sox9 (NM_{—}000346) and Collagen type-II (NM 001844.4). Each sample was analysed in triplicate. The PCR reaction was initiated by a 2 minute 50° C. and 10 minute 95° C. step to optimise thermal cycling conditions for the ABI Prism 7700 sequence detection system (Applied Biosystems, USA) used to detect relative quantification of gene expression. This was followed by PCR amplifications performed for 40 cycles in a Corbett Rotor-Gene 6000 (Corbett Life Science, Australia) at 95° C. for 15 seconds and 60° C. for 1 minute. The target signal was plotted against the number of cycles and the threshold level was set at 0.05. Comparison of all data was taken at the intercept, where sample reactions crossed this phase of amplification. Our results were correlated using the comparative C_{T} method (Wang et al., 2006, J. Assoc. Lab. Aut., 11, 314-318). Fold changes in gene expression were presented as mean±standard deviation change relative to day 0 cells. The relative expression level for each target gene was normalized by the Ct value for the housekeeping gene 18S (X03205).

Statistical Analysis

The means and standard deviations of the results were calculated using the SPSS 12.0 software package (SPSS Inc., Chicago, USA). The Mann-Whitney U Test for two independent samples was performed to determine statistical significance between various scaffolds. A p value<0.05 was considered to be statistically significant.

Results and Discussion

FIG. 1 show a typical fibrous scaffold used in this invention. Electrospun fibres resemble the nanosize-scale of fibres from the cartilage extracellular matrix. The fibre diameter distribution is quite narrow and average fibre diameter is 242 nm as calculated from the measurement of 40 fibres per image of sample (in triplicate).

The surface atomic composition (carbon, oxygen, nitrogen) of plasma treated PLLA electrospun samples with different NH₃ gas exposure times is presented in FIG. 2-A. The spectra show three main signals corresponding to C is (285 eV) 0 is (532 eV) and N 1 s (400 eV). Corrected chemical composition calculated from the relative areas of the XPS spectra of different samples shows that the amount of nitrogen species on the surface increases up to 5.2 atomic %, corresponding to a power supply of 100 W and 10 minutes exposure (FIG. 2-B). The peak at 399.7 eV was assigned to —N═H— (Yang et al., 2002. Biomaterials, 2607-2614). Additionally, untreated PLLA and NH₃ plasma treated PLLA were analysed by ATR-FITR (FIG. 3) to evaluate the presence of amine groups. In both samples the presence of typical PLLA peaks at 1754 cm⁻¹ (vC═O), 1450 cm⁻¹ (δₐₛC—H) and 1085 cm⁻¹ (vₛC—O—C) were observed in accordance with other authors findings (Paragkumar et al., 2006. Appl. Surf. Sci., 253, 2758-2764; Kister et al., 1999. Polymer, 39, 2, 267-273). The plasma treated PLLA spectra also shows a weak broad band in the 3400-3200 cm⁻¹ region and a weak shoulder in the 1650-1550 cm⁻¹ region which indicate the presence of N—H stretching and N—H bending vibrations. Chemical quantification using the ninhydrin assay showed that the density of primary amine groups on the ammonia plasma treated PLLA surface increased with longer exposure time and higher power (FIG. 2-C). The densities of amine groups on the surface modified scaffolds were deduced from the standard curve with the highest density corresponding to plasma treatment at 100W for 10 minutes (66.42 nmol/mg PLLA).

The presence of nitrogen species on the surface of the plasma treated PLLA shown by XPS analysis, combined with results from ATR-FTIR and ninhydrin demonstrated the incorporation of amine groups onto the surface of plasma treated PLLA. These amine groups were available for the subsequent conjugation step.

The results of the TGF-β1 immunoassay showed that the LTGF-β1 complex was successfully immobilized onto the electrospun scaffold surfaces using either the random or the oriented approach, although in different amounts. An average of 195.4±34 pg/cm² of TGF-β1 were activated from the pt-LTGF scaffolds whereas 14.1±1.7 pg/cm² were recovered from the sLTGF group. No TGF-β1 was detected on the rest of the groups.

FIG. 4 shows the results from the Live/Dead assay after 1, 7 and 14 days of cell seeded scaffolds in culture. Good retention of cells on the scaffold is a critical issue for clinical application/transplantation. After 14 days, cell viability of human articular chondrocytes on plasma treated PLLA (ptPLLA) drops from 100% to around 65% whereas in scaffolds modified with recombinant LTGF cell viability was maintained above 90%.

MTS assay was used to compare cell proliferation on different modified scaffolds based on the detection of metabolic activity. FIG. 5 show the results from the MTS proliferation assay after 1, 7 and 14 days of seeded scaffolds in culture. Metabolic activity was significantly higher (p<0.01) on cells cultured on pLTGF (LTGF randomly linked to plasma treated PLLA)-and sLTGF (LTGF oriented immobilized on ptPLLA modified with Sulfo-SMCC) scaffolds than on plasma treated ptPLLA (p<0.012). Furthermore there was a statistically significant (p<0.015) difference between cells cultured on scaffolds with randomly immobilized LTGF in comparison with cells cultured on scaffolds with LTGF immobilized in an oriented manner using Sulfo-SMCC at days 7 and 14.

FIG. 6 shows a SEM image of articular chondrocytes interacting with sLTGF modified PLLA fibres. Cells can be seen attached and spread on the scaffold.

The mRNA expression of cartilage-specific and dedifferentiation markers for the nasal chondrocytes cultured on the various scaffold types were analysed using real-time RT-PCR (FIG. 8). An existing problem with the generation of cartilage substitutes is the maintenance of the chondrogenic phenotype. Chondrocytes rapidly dedifferentiate, expressing fibroblastic markers (such as Col1A) whilst losing expression of cartilage-specific genes such as Sox9. The results shown herein demonstrate that latent TGF-β1 functionalised scaffolds (pLTGF) significantly up-regulated the Sox9 expression by approximately 10-fold when compared to day 0, indicating the maintenance of the differentiated chondrocyte phenotype. The random orientation of latent TGF-β on the biomaterial clearly did not compromise the bioavailability of TGF to the seeded and newly grown cells. This level of expression was also significantly higher than all other scaffold groups in this experiment. Col1A1, a dedifferentiation marker for chondrocytes, was significantly higher in the TGF group, as compared to other scaffold groups.

We also considered the efficacy of the TGF-β1 in the induction of differentiation per nanogram of the growth factor in each group—by dividing the gene expression data by the amount of TGF-β1 supplemented to media (TGF group) or present on the functionalised scaffolds (pLTGF and sLTGF groups) (FIG. 9). The sLTGF group was more effective in inducing a chondrocytic differentiation if we consider the effectiveness per nanogram of TGF-β1. Thus, the effectiveness of the biomaterials of the invention can be further improved by optimising the method of immobilising specifically oriented latent TGF-β onto the biomaterial so that more latent TGF-β is immobilised. Such optimisation may include, for example, the use of a longer intermediate molecule linking the latent TGF-β complex to the biomaterial, the thiol-specific modification of the SLC molecule and purification before linking to the scaffold, and other methods known in the art. Biomaterials having more specifically oriented immobilised latent TGF-β will result in a greater, sustained level of differentiation of cells into chondrocytes.

Taken together, our findings demonstrate that LTGF-modified fibres influence cell behaviour of seeded human chondrocytes in vitro isolated from articular and nasal cartilage. The results provide evidence that the immobilized LTGF keeps its bioactivity after 14 days in cell culture medium. These results show that this new method of presenting a latent form of TGFβ1 to cells has a valuable application in vitro and in vivo in cartilage regeneration and also in bone and many other tissues where TGFβ1 has an important role (see above).

1. A biomaterial having a latent form of a growth factor immobilised thereon.

2. The biomaterial of claim 1, wherein the latent form of the growth factor comprises a growth factor associated with a latency associated peptide.

3. The biomaterial of claim 1, wherein the growth factor is TGF-β1 TGF-β2, TGF-β3, TGF-β4, TGF-β5 or any other member of the TGF-β superfamily.

4. The biomaterial of claim 1, further comprising cells thereon.

5. A method of producing the biomaterial of claim 1, comprising immobilising the latent form of the growth factor on the biomaterial.

6. The method of claim 5, comprising:
modifying the biomaterial to provide functional groups which are capable of binding to functional groups on the latent form of the growth factor; and
contacting the modified biomaterial with the latent form of the growth factor under conditions which allow the latent form of the growth factor to bind to the functional groups on the biomaterial.

7. The method of claim 6, wherein the biomaterial is modified with amine groups and/or maleimide groups which are capable of binding with functional groups on the latent form of the growth factor.

8. The method of claim 6, wherein the biomaterial is modified with an antibody specific for the latent form of the growth factor.

9-11. (canceled)

12. A method of treating tissue damage in a patient, comprising implanting the biomaterial of claim 1 into the patient.

13. The biomaterial of claim 3, wherein the any other member of the TGF-β superfamily is an activin, inhibin, or bone morphogenetic protein.

14. The biomaterial of claim 13, wherein the bone morphogenetic protein is BMP1, BMP2, BMP3, BMP4, BMP5, BMP6, or BMP7.
